(19) United States
(12) Patent Application Publication
(10) Pub. No.: US 2002/0022836 A1
(43) Pub. Date: Feb. 21, 2002

(54) Electrosurgery system

(75) Inventors: Colin Charles Owen Goble, Surrey (GB); Nigel Mark Goble, Berkshire (GB); Francis Amoah, Cardiff (GB)

Correspondence Address: OLIFF & BERRIDGE, PLC, P.O. BOX 19928, ALEXANDRIA, VA, 22320, US

(73) Assignee: GYRUS MEDICAL LIMITED, Cardiff (GB)

(21) Appl. No.: 09/945,833

(22) Filed: Sep. 5, 2001

Related U.S. Application Data
(63) Continuation-in-part of application No. 09/517,631, filed on Mar. 3, 2000.
(60) Provisional application No. 60/229,537, filed on Sep. 5, 2000.
(60) Provisional application No. 60/141,261, filed on Jun. 30, 1999.

(30) Foreign Application Priority Data
Mar. 5, 1999 (GB) 9905210.2

(51) Int. Cl. A61B 18/12; A61B 18/14

(52) U.S. Cl. 606/34; 606/39; 606/41; 606/45

(57) ABSTRACT

An electrosurgery system for electrosurgically cutting or vaporising living tissue includes an electrosurgical generator having a pair of output terminals coupled to an electrosurgical instrument containing an electrode assembly. The electrode asssembly has at least one treatment electrode and an adjacent return electrode. The generator and the assembly are arranged to deliver to the treatment and return electrodes radio frequency (r.f.) energy individually or simultaneously at at least two frequencies, one of which is below 100 MHz and the other of which is above 300 MHz. The generator includes a load-responsive control circuit which, in one mode, causes power to be generated predominantly at the lower frequency when the load impedance is high and predominantly at the upper frequency when it is low. This allows automatic switching between cutting and coagulation operation. In another embodiment the r.f. current delivered at the lower frequency is limited in order to restrict dissipation of power in the tissue at that frequency and to permit tissue cutting or vaporisation using energy delivered simultaneously at the higher frequency. In yet another embodiment, the instrument includes a gas plasma generator operating such that an ionisable gas is energised in a gas supply passage by the upper frequency component to form a plasma stream which acts as a conductor for delivering the lower frequency component to a tissue treatment outlet of the passage.

### RELATED APPLICATION

This application is a continuation-in-part of application Ser. No. 09/517,639 filed Mar. 3, 2000.

### FIELD OF THE INVENTION

This invention relates to a radio frequency electrosurgery system and associated methods of operation.

### BACKGROUND OF THE INVENTION

It is known to use a needle or narrow rod electrode for cutting tissue in monopolar electrosurgery at frequencies in the range of 300 kHz to 3 MHz. An electrosurgical signal in this frequency range is applied to the electrode, and the electrical current path is completed by conduction through tissue to an earthing plate secured to the patient's body elsewhere. The voltage applied to the electrode must be sufficiently high to cause arcing and consequent thermal rupture so that tissue adjacent the needle is ablated or vaporised.

At lower power levels, coagulation of the tissue can be achieved, i.e. without arcing, due to thermal dissipation of energy in the tissue adjacent the electrode. However, with a narrow electrode as commonly used for tissue cutting, desiccation of the tissue immediately adjacent the electrode and build-up of desiccated material on the electrode itself constitutes a high-impedance barrier to further coagulation. Spatula-shaped electrodes have been produced to overcome the difficulty in providing a dual-purpose electrode, i.e. one suitable for both cutting and coagulation. The designer's intention is that the edge of the electrode is used for cutting, whereas the flat surface is used for coagulation. However, coagulation with such an electrode tends to be imprecise due to the size of the flat surface, with the result that a large thermal margin is produced.

It is an object of the invention to provide a means of achieving both tissue cutting and coagulation with a single electrode assembly.

### SUMMARY OF THE INVENTION

According to one aspect of this invention, there is provided an electrosurgery system comprising an electrosurgical generator, a feed structure and an electrode assembly, the electrode assembly having at least one active electrode and at least one adjacent return electrode each of which is coupled to the generator via the feed structure, wherein the generator and feed structure are capable of delivering radio frequency (r.f.) power to the active and return electrodes in lower and upper frequency ranges simultaneously, and wherein the lower frequency range is below 100 MHz and the upper frequency range is above 300 MHz. The lower frequency range may extend upwardly from 100 kHz and is preferably 300 kHz to 40 MHz. The upper frequency range may extend from 300 MHz to 10 GHz, preferably above 1 GHz, with operating frequencies in the upper and lower ranges having a frequency ratio of 5:1 or greater. Typically, the generator is arranged such that the r.f. power delivered in the upper frequency range is at a fixed frequency which is at least ten times the frequency of power delivered in the lower frequency range. Indeed, a fixed frequency of 2.45 GHz in the upper frequency range is preferred.

The system allows simultaneous delivery of lower and upper frequency range components to the electrodes. In one embodiment it is possible to provide a combination of medium or low frequency tissue cutting, vaporisation or ablation together with coagulation of surrounding tissue to a degree dependent upon the amplitude of the component in the upper frequency range. This embodiment may be used for tissue cutting, vaporisation or ablation in a monopolar mode, with a separate earthing electrode applied to the outside of the patient's body. Coagulation can occur in a quasi-bipolar mode whereby the return current path in the upper frequency range runs from the tissue adjacent the operation site to the return electrode of the electrode assembly due to capacitive coupling. It will be understood that the system may allow selection of power delivery either in the lower frequency range or the upper frequency range depending upon the kind of treatment required. This selection may be performed manually by the surgeon or automatically in a manner described below. In addition, power may be supplied in both frequency ranges simultaneously to obtain a blended cutting and coagulation effect, the two components being linearly added or otherwise combined in a single signal feed structure.

In this embodiment of the invention, the generator includes a control circuit responsive to electrical load and operable to cause the delivered power to have a predominant frequency component in the lower frequency range when the load impedance is in an upper impedance range, and to have a predominant frequency component in the upper frequency range when the load impedance is in a lower impedance range. In this way, it is possible to cut, ablate or vaporise living tissue (i.e. causing cell rupture) with the lower frequency range component but also to bring about efficient coagulation when a very low load impedance is detected, indicating the presence of electrolytic fluid such as blood from a blood vessel, requiring coagulation. The system reverts to predominantly low frequency operation once the impedance has risen above a predetermined threshold following coagulation.

When electrical load impedance is used as the control stimulus, a signal representative of load impedance being compared with a reference signal, the reference signal may have different levels depending on whether the generator is to be switched from a predominant low frequency component to a predominant high frequency component or vice versa. In other words, different load impedance thresholds may be selected when operating in the lower frequency range or the upper frequency range respectively.

A composite signal having components from both frequency ranges may be produced by combining (e.g. adding) the signals from two generator stages, one operating in the region of, say, 1 MHz and the other operating at 2.45 GHz. Both generator stages may be in a single supply unit coupled to an electrosurgical instrument which consists of a handpiece mounting the electrode assembly so that, for instance, the two frequency components are fed from the supply unit to the handpiece by common delivery means such as a low loss flexible coaxial cable. Alternatively, the generator stage producing the UHF frequency component may be located in the handpiece to reduce transmission losses and radiated interference, the signal combination being performed within the handpiece as well.

Typically, the electrode assembly is at the distal end of a rigid or resilient coaxial feed forming the above-mentioned feed structure. To reduce extraneous UHF radiation, an isolating choke element in the form of a conductive quarter-wave stub or sleeve may be mounted to the outer supply conductor of the coaxial feed in the region of the distal end. The active electrode may take the form of a rod or pin projecting from the coaxial feed distal end. The return electrode may be a conductive sleeve, plate or pad connected to the outer supply conductor at the feed distal end and extending proximally over the outer conductor but spaced from the latter so that the active electrode rod and the return electrode sleeve, plate or pad together form an axially oriented dipole at the operating frequency of the generator in the upper frequency range. Alternatively, the return electrode simply takes the form of a distal end portion of the feed outer conductor located distally of the choke. The return electrode may be covered with an electrically insulative layer in order that, when the active electrode is applied to tissue, the return electrode, being set back from the active electrode so as normally to be spaced from the tissue, acts as a capacitive element forming part of a capacitive return path between the treated tissue and the return supply conductor of the feed.

According to another aspect of the invention, an electrosurgery system for electrosurgically cutting or vaporising tissue comprises an electrosurgical generator and an electrode assembly having at least one treatment electrode and an adjacent return electrode, wherein the generator and the assembly are arranged to deliver to the treatment and return electrodes radio frequency (r.f.) energy simultaneously at at least two frequencies. One of the frequencies is in a lower frequency range of from 50 kHz to 50 MHz and the other is greater than 300 MHz. The r.f current delivered in the lower frequency range is limited such that the current-to-frequency ratio of energy delivered in the lower frequency range remains below a value of 17 mA r.m.s. per 100 kHz. In this way it is possible to strike an arc between the treatment electrode and the tissue to be treated using the r.f. energy in the lower frequency range, this arc providing a low impedance pathway for energy at a frequency greater than 300 MHz to cause cell rupture and, as a result, cutting or vaporisation of the tissue. The return path for energy at the higher frequency is predominantly through the stray capacitance between the tissue and the return electrode. This is particularly the case for current at the frequency greater than 300 MHz. One of the effects of this is that tissue outside the treatment site is substantially unaffected. Since the arc is established using low frequency energy, the components for generating and transmitting energy at the higher frequency, i.e. above 300 MHz, may be designed solely to drive a low impedance. Furthermore, since coagulation of tissue generally requires high current, and the tissue presents a low impedance to the source, the electrode assembly may be constructed to provide UHF matching into a low impedance load, the system thereby providing efficient operation in both cutting/vaporisation and coagulation modes using the single electrode assembly. Since the capacitive pathway from tissue to return electrode is of considerably lower impedance than at the lower frequency, high current can be delivered at UHF, the current density necessary for tissue treatment being confined to the treatment area. Tissue effects due to the low frequency energy are minimal due to the restriction of low frequency currents to low levels.

One of the ways of restricting low frequency current is to ensure that the source impedance at the operating frequency in the lower frequency range is comparatively high. The preferred system comprises a generator unit having a pair of r.f. output terminals, an instrument which includes a handpiece, a shaft mounted on the handpiece and the electrode assembly generally located at a distal end of the shaft, and a feeder cable arranged to connect the generator unit output terminals to the handpiece. The preferred lower frequency range is 100 kHz to 5 MHz. The high source impedance may be achieved by connecting a low value capacitor in series in the low frequency current path, e.g. between the feeder cable and the treatment electrode for restricting the current at the lower operating frequency such that the current-to-frequency ratio remains within the range referred to above. Preferably, the capacitor is located at the distal end of the shaft, immediately adjacent the treatment electrode. The instrument shaft may comprise a pair of supply conductors for delivering the r.f. energy to the electrode assembly, the capacitor being formed as the coaxial combination of a elongate inner conductor which is integrally formed with the treatment electrode, and a tubular outer conductor spaced from the inner conductor by a tubular heat resistant dielectric tube, this tubular outer conductor being connected to one of the supply conductors of the shaft. Typically, in this case, the capacitor has a value of 5 pF or less.

At UHF, the reactance of the capacitor is low and, therefore, has little effect on the transmission of UHF power to the treatment electrode.

The instrument shaft preferably includes a balun, advantageously mounted close to the electrode assembly. Such a balun, being configured to operate at the higher operating frequency serves to improve efficiency and to minimise tissue effects outside the treatment area.

It is also possible to raise the source impedance and hence limit the low frequency output current by arranging for the low frequency source in the generator unit to drive a resonant load, e.g. in the form of a shunt parallel resonant circuit with a Q in the region of 100 or greater. The parallel capacitance may be the capacitance of the feeder between the generator unit and the handpiece, while the parallel inductance, tuning the capacitance to the lower of the operating frequencies is preferably situated inside the generator unit and upstream of the stage performing combination of the high and low frequency signals. The resonant circuit allows voltages in excess of 700 V to be generated, allowing formation of an arc between the treatment electrode and the tissue being treated. When the arc is struck or the treatment electrode touches tissue, the Q of the resonant circuit is reduced, and the output voltage collapses to prevent current delivery beyond the range specified above. The low frequency signal may be pulsed. This allows the driving impedance of the low frequency source into the resonant circuit to be reduced without exceeding the average current-to-frequency ratio. This in turn allows the rise time of the low frequency output voltage to be increased, despite the presence of the resonant circuit.

Whether the low frequency signal is continuous or pulsed, the maximum r.f. power delivered, continuously or during each r.f. burst, respectively, is preferably limited to 10 W or less. The output voltage of the low frequency source may also be limited.

As a further alternative, the low frequency source impedance may be increased by inserting a series impedance such as a resistance in the low frequency output current path in a low frequency part of the generator unit.

According to a further aspect of the invention, a method of operating an electrosurgical tissue cutting or vaporisation system which comprises an electrosurgical instrument having an active electrode and an adjacent return electrode, comprises supplying to the electrodes radio frequency (r.f.) energy simultaneously at at least two frequencies, one of which is in a lower frequency range of 50 kHz to 50 MHz and the other of which is greater than 300 MHz, the current in the lower frequency range whilst the instrument is set to operate in a tissue cutting or vaporising mode being such that the current-to-frequency ratio of energy delivered in the lower frequency range remains below a value of 17 mA r.m.s. per 100 kHz.

According to yet a further aspect of the invention, a method of electrosurgically cutting or vaporising tissue using an electrosurgery system which comprises an electrosurgical generator and an electrode assembly having at least a treatment electrode and an adjacent return electrode, comprises bringing the treatment electrode to a position on or adjacent the tissue to be cut or vaporised, applying to the electrodes a first radio frequency (r.f.) signal component at least one frequency in the range of from 50 kHz to 50 MHz to establish an arc between the treatment electrode and the tissue, and simultaneously applying to the electrodes a second r.f. signal component at at least one second frequency which is greater than 300 MHz to cause a current at the second frequency to flow along the arc established by the first r.f. signal component, the level of the average current above 300 MHz being at least an order of magnitude greater than the average current in the frequency range of from 50 kHz to 50 MHz during a cutting or vaporisation operation.

Preferably, the average current in the frequency range of from 50 kHz to 50 MHz is small enough to have no clinical effect or negligible total effect in the absence of the second r.f. signal component. A maximum value below 50 mA is typical.

In an alternative embodiment in accordance with the invention, the electrode assembly includes a gas supply passage and the active electrode is located within the passage where it acts as a gas-ionising electrode. In this case, the active electrode acts as a low- to high-impedance transformer at the operating frequency of the generator in the upper frequency range, producing an intense electric field in the space between the distal end portion of the active electrode and the return electrode. Accordingly, when there is an ionisable gas in the passage, the major part of the power delivered to the electrode assembly in the upper frequency range is dissipated in the passage. In the lower frequency range no transforming effect occurs and the frequency component in the lower frequency range is, instead, delivered to the tissue to be treated by the ionised gas plasma which, in effect, acts as a monopolar gaseous electrode. Use of a UHF frequency component as a plasma generator and a lower frequency component for electrosurgery allows independent control of plasma generation and electrosurgical power delivery, thereby avoiding the disadvantage of known single r.f. source gas plasma electrosurgery devices. Typically, in such a prior device the ability of the source to deliver current through the plasma is severely hampered due to the requirement for high peak voltages when using low frequencies (i.e. typically, less than 1 MHz).

### BRIEF DESCRIPTION OF THE DRAWINGS

The invention will be described below by way of example and with reference to the drawings. In the drawings:

FIG. 1 is a diagram showing an electrosurgical system in accordance with the invention;

FIG. 2 is a diagrammatic cut away perspective view of an electrode assembly and associated feed structure;

FIG. 3 is a diagram showing a simulation of the electric field pattern obtainable with the electrode assembly of FIG. 2;

FIG. 4 is an electrical block diagram of the system of FIG. 1;

FIG. 5 is a circuit diagram of a low frequency output circuit which may be used in the generator shown in FIG. 4;

FIG. 6 is a graph showing the variation of delivered power and voltage obtained from the low frequency generator part of FIG. 5;

FIG. 7 is a circuit diagram of a generator control circuit;

FIG. 8 is a microstrip layout for a mixer adding the signals obtained from low and high frequency parts of the generator;

FIG. 9 is a circuit diagram for a power control circuit forming a portion of a high frequency generator part;

FIG. 10 is a cross-section diagram of an alternative electrode assembly configured for gas plasma generation; and

FIG. 11 is a cross-section diagram of a further alternative electrode assembly configured for gas plasma generation.

FIG. 12 is a block diagram of a modified electrosurgical system in accordance with the invention;

FIGS. 13A and 13B are perspective views of a UHF tissue vaporising instrument, FIG. 13A being partly cut away;

FIGS. 14A and 14B are perspective views of a UHF tissue cutting instrument, FIG. 14A being partly cut away;

FIG. 15 is an equivalent circuit diagram of the instruments of FIGS. 13A, 13B, 14A and 14B;

FIG. 16 is a low frequency load curve showing raised source impedance; and

FIGS. 17A and 17B are simplified circuit diagrams of low frequency energy supply circuits in alternative electrosurgical systems in accordance with the invention.

### DETAILED DESCRIPTION OF THE PREFERRED EMBODIMENTS

The preferred embodiments of the present invention are applicable mainly to the performance of electrosurgery upon tissue in a gaseous environment using a dual electrode instrument having active and return electrodes situated at the distal end of an instrument shaft. The active electrode is applied directly to the tissue. The return electrode does not contact the tissue being treated, but is normally adjacent the tissue surface where it is capacitively coupled to the tissue at UHF frequencies.

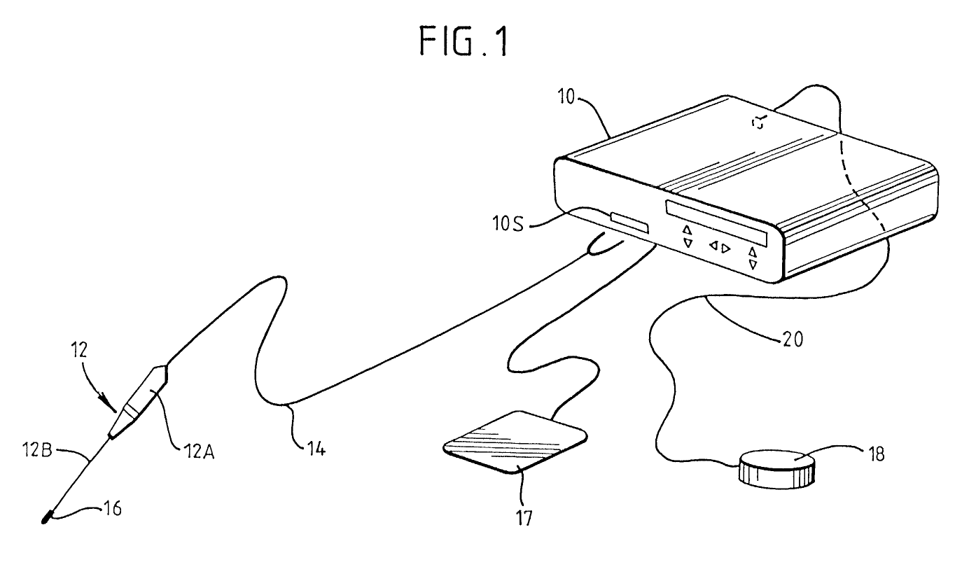

One system incorporating such an instrument is shown in FIG. 1. Referring to FIG. 1, the system has a electrosurgical supply unit 10 with an output socket 10S providing a radio frequency (r.f.) output for the electrosurgical instrument 12 via a flexible cable 14. Instrument 12 has a handpiece 12A and, mounted to the handpiece, an instrument shaft 12B having an electrode assembly 16 at its distal end. A patient return pad 17 is also connected to the supply unit 10 in this embodiment of the invention. Activation of the supply unit may be performed from the handpiece 12A via a control connection in cable 14, or by means of a foot switch 18 connected separately to the rear of the supply unit 10 by a foot switch connection cable 20.

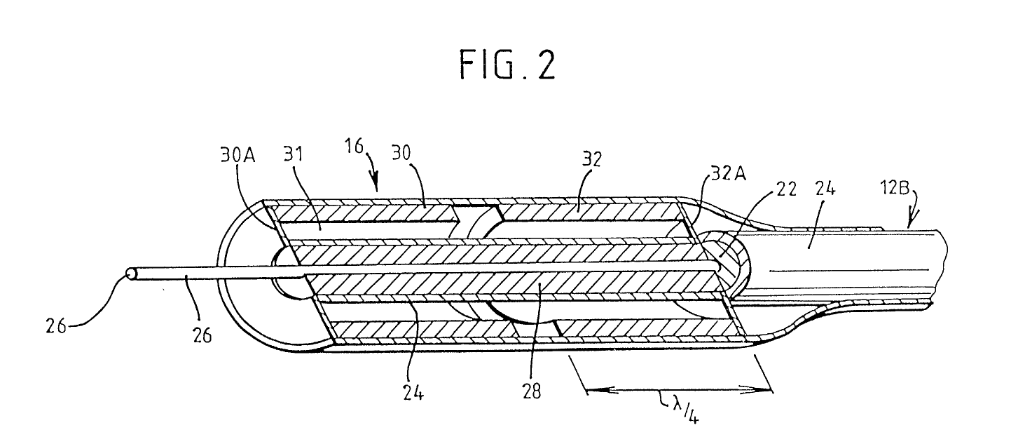

Instrument shaft 12B constitutes a feed structure for the electrode assembly 16 and takes the form of a rigid coaxial feed having an inner conductor and an outer supply conductor made with rigid material constructed as a resilient metal tube or as a plastics tube with a metallic coating. The distal end of the feed structure appears in FIG. 2 from which it will be seen that the inner conductor 22 has an extension which projects beyond the outer conductor 24 as a rod 26 forming an axially extending active electrode of a diameter typically less than 1 mm. Where they are surrounded by the outer supply conductor 24, the inner supply conductor 22 and the active electrode 26 are encased in a coaxial ceramic or high-temperature polymer sleeve 28 acting as an insulator and as a dielectric defining the characteristic impedance of the transmission line formed by the coaxial feed.

The return electrode is formed as a coaxial conductive sleeve 30 surrounding a distal end portion of the outer supply conductor 24 with an intervening annular space 31. An connection between the return electrode 30 and the outer supply conductor 24 is formed as an annular connection 30A at one end only, here the distal end, of the return electrode 30 such that the projecting portion of the active electrode 26 and the return electrode 30 together constitute an axially extending dipole with a feed point at the extreme distal end of the coaxial feed. This dipole 26, 30 is dimensioned to match the load represented by the tissue and air current path to the characteristic impedance of the feed at or near 2.45 GHz.

Located proximally of the electrode assembly formed by active electrode 26 and return electrode 30 is an isolating choke constituted by a second conductive sleeve 32 connected at one of its ends to the outer supply conductor 24 by an annular connection 32A. In this instance, the annular connection is at the proximal end of the sleeve. The sleeve itself has an electrical length which is a quarter-wavelength (λ/4) at 2.45 GHz or thereabouts, the sleeve thereby acting as an balun promoting at least an approximately balanced feed for the dipole 26, 30 at that frequency.

The projecting part of the active electrode 26 has a length in the region of 10 mm while the return electrode 30 is somewhat greater than 10 mm in length. The reason for this difference in length is that the relative dielectric constant of living tissue is higher than that of air, which tends to increase the electrical length of the active electrode for a given physical length. The electrode assembly 16 and choke 32 are configured to provide an electrical impedance match with the tissue being treated and, advantageously, a mismatch to the impedance of free space, so that power transmission from the electrode assembly is minimised when the active electrode is removed from tissue whilst an electrosurgical voltage is still being applied at 2.45 GHz.

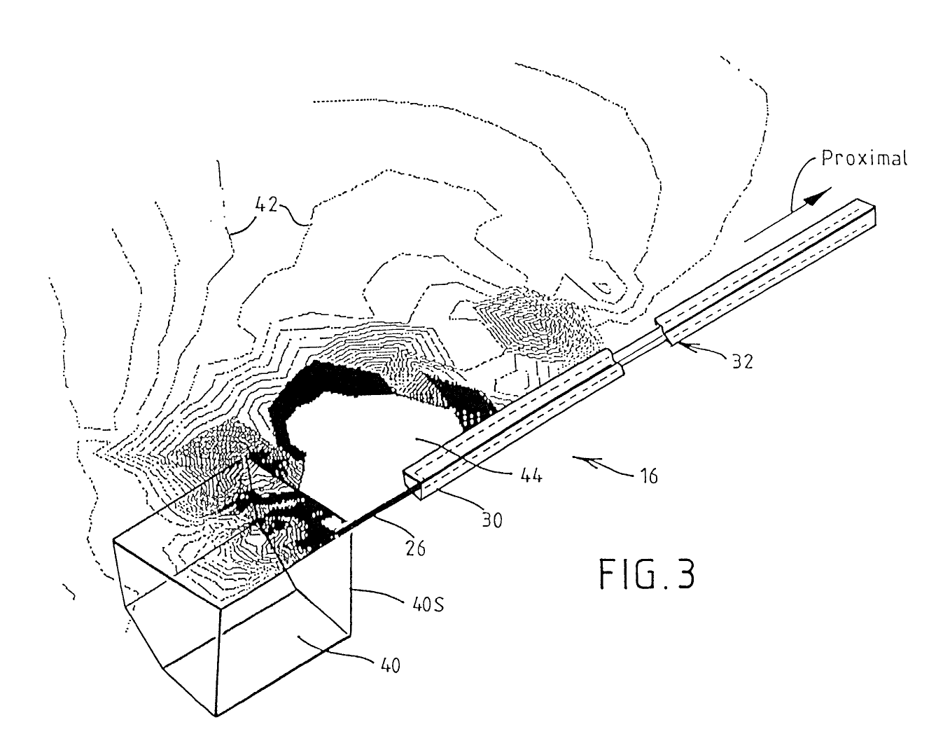

Sleeve 32 has an important function insofar as it acts as an isolating trap isolating the outer supply conductor 24 of the feed structure from the return electrode 30, largely eliminating r.f. currents at 2.45 GHz on the outside of the outer supply conductor 24. This also has the effect of constraining the electric field which results from the application of a voltage at 2.45 GHz between the active electrode and the return electrode, as seen in FIG. 3.

FIG. 3 is a computer-generated finite element simulation of the electric (E) field pattern produced by the electrode assembly 16 and choke 32 of FIG. 2 when energised via the coaxial feed 12B at 2.45 GHz. It should be noted that the components of the electrode assembly and the sleeve 32 are shown quartered in FIG. 3 (i.e. with a 90° sector cross-section). The active electrode 26 is shown with its tip in contact with a body 40 of tissue. The pattern 42 of E-field contours in a plane containing the axis of the electrode assembly illustrates the marked concentration of E-field in the space 44 surrounding the active electrode 26 and the distal part of the return electrode 30 immediately adjacent the tissue surface 40S. Proximally of this space, the E-field intensity is much reduced, as will be seen by the relatively wide spacing of the contours. (It should be noted that the region 44 of greatest intensity appears as a white area in the drawing. In this region and the immediately surrounding region the contour lines are too closely spaced to be shown separately.) The presence of an intense E-field region between the distal end of the return electrode 30 and the tissue surface 40S is also indicative of capacitive coupling between these two surfaces at the frequency of operation (which is 2.45 GHz in the simulation of FIG. 3). Localisation of the E-field in this manner also has the effect of reducing radiated loss in comparison with an arrangement in which intense field regions exist further from the tissue surface 40S, with the effect that radiated loss is minimised.

Referring back to FIG. 2, it will be understood that the feed structure makes use of a coaxial feed rather than a waveguide to transmit power to the electrode assembly from the handpiece and, indeed, as shown in FIG. 1, there is a flexible cable between the handpiece 12 and the electrosurgical supply unit 10. Use of coaxial feeders rather than waveguides in both cases allows the transmission of voltage components of widely spaced frequencies in a single transmission line. This also provides the advantage of a flexible connection between the handpiece 12 and the supply unit 10. Dielectric losses in the cable 14 are mitigated by selection of a cable with a low density, partly air-filled dielectric structure. A further reduction in dielectric loss can be obtained by increasing the diameter of the cable. Such increased diameter need not be used over the whole length of the cable 14. Indeed, a smaller diameter may be retained near the handpiece to retain flexibility of movement.

The ability to feed different voltage components at different frequencies from the supply unit to the handpiece in a single transmission line has advantages related to the main aspect of the present invention which is the provision of means for delivering r.f. power to the electrode assembly in lower and upper frequency ranges, the upper range containing frequencies at least five times the frequencies of the lower frequency range. Thus, the supply unit may include generator parts generating electrosurgical signals at, for instance, 1 MHz and 2.45 GHz respectively to suit different operation site conditions and surgical requirements. In the preferred embodiments of the invention, these different components are supplied simultaneously through cable 14 to the handpiece 12 and electrode assembly 16.

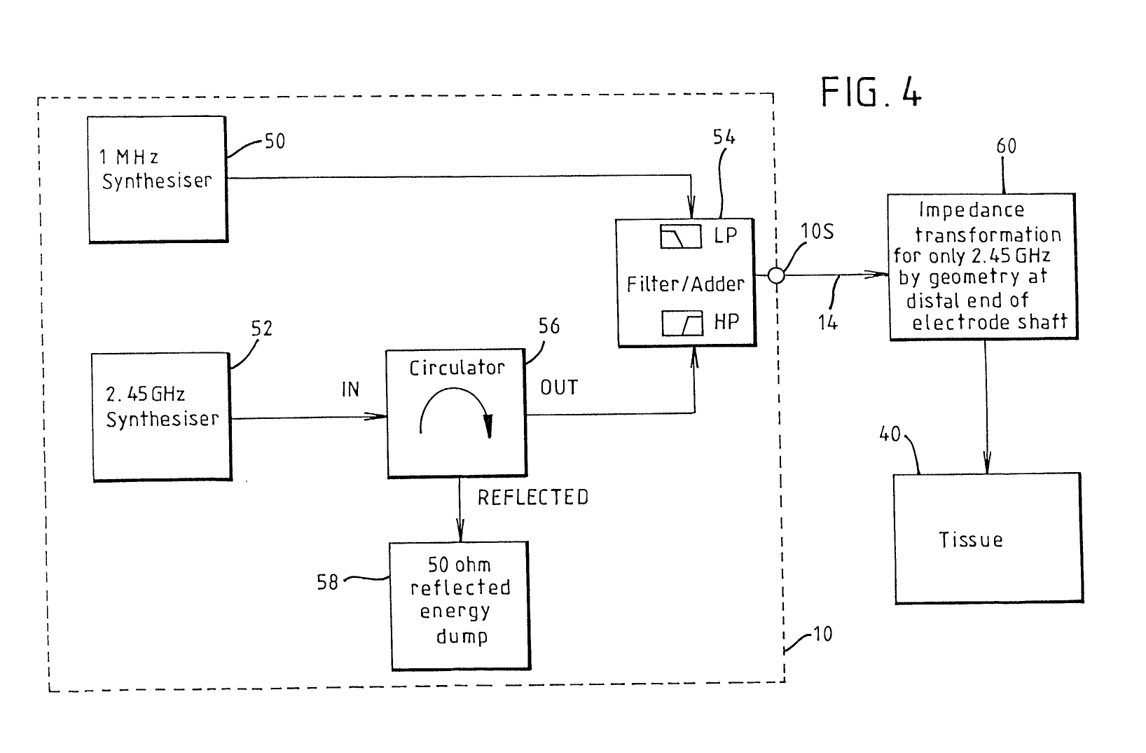

Details of an electrosurgical generator for delivering electrosurgical power in this way will now be described with reference to FIGS. 4 to 9.

Referring to FIG. 4, the supply unit 10 contains separate 1 MHz and 2.54 GHz synthesisers 50, 52 the output signals of which are summed in an adder stage 54 having low- and high-pass filters coupled to inputs arranged to receive the 1 MHz and 2.45 GHz signals respectively, as shown. A circulator 56 connected in series between the 2.45 GHz synthesiser 52 and the adder 54 serves to provide a 50 ohm source impedance for synthesiser 52 under conditions of varying load impedance, reflected power being dissipated in a 50 ohm reflected energy sink or dump 58, also connected to the circulator 56.

At the output of the adder 54 a composite signal consisting principally of the two components at 1 MHz and 2.45 GHz is delivered to the output socket 10S of the supply unit and thence via cable 14, which is typically in the region of three meters long, to the handheld instrument, represented in FIG. 4 by an impedance transformer 60 operable at 2.45 GHz, and thereafter to the tissue 40 under treatment.

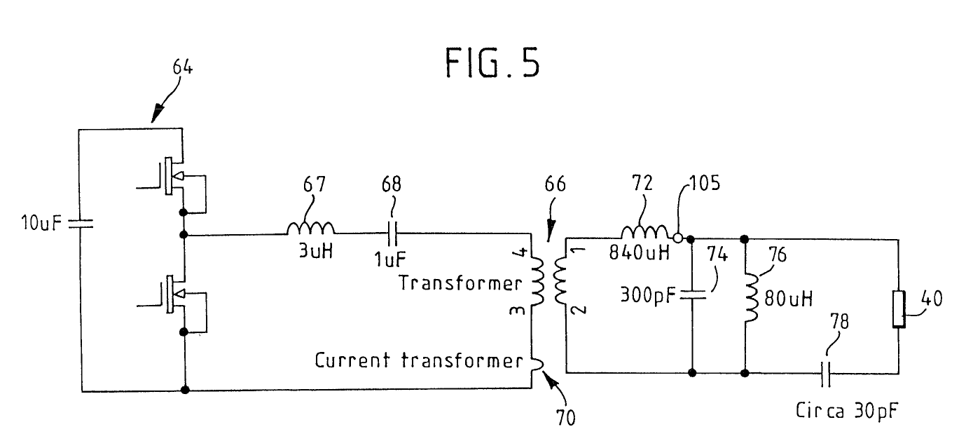

Referring to FIG. 5, the 1 MHz synthesiser has a push-pull output stage 64 which drives an output transformer 66 via a current limiting inductor 67 of 3 μH and a series coupling capacitor 68 of 1 μF. Included in the primary circuit of the transformer 66 is a shunt current transformer 70 having an output winding (not shown) for monitoring the output current of the synthesiser at 1 MHz. The transformer secondary winding is coupled to the output 10S through a tuning inductance 72 of 840 μH which resonates with the capacitance of the cable 14 and other components on the secondary side of the transformer 66. In this example the cable has an inherent shunt inductance of about 80 μH and the series capacitance 78 between the return electrode and the tissue being treated is in the region of 30 pF. The tissue is shown as a resistance 40. Those skilled in the art will understand that at 1 MHz, series inductance 72 and capacitance 78 can resonate so as to act as a short circuit, thereby coupling the load (tissue resistance 40) directly to the transformer secondary under matched conditions. The effect of the series inductance 67 in the primary circuit is to limit the secondary current at 1 MHz typically to 50 mA. The capacitance 78 is larger than 30 pF of the patient-attached return pad 17 (see FIG. 1) is used such that, at 1 MHz, the system is used in a monopolar mode.

It will be understood that the filter/adder circuitry shown in FIG. 4 has been omitted from FIG. 5 for clarity.

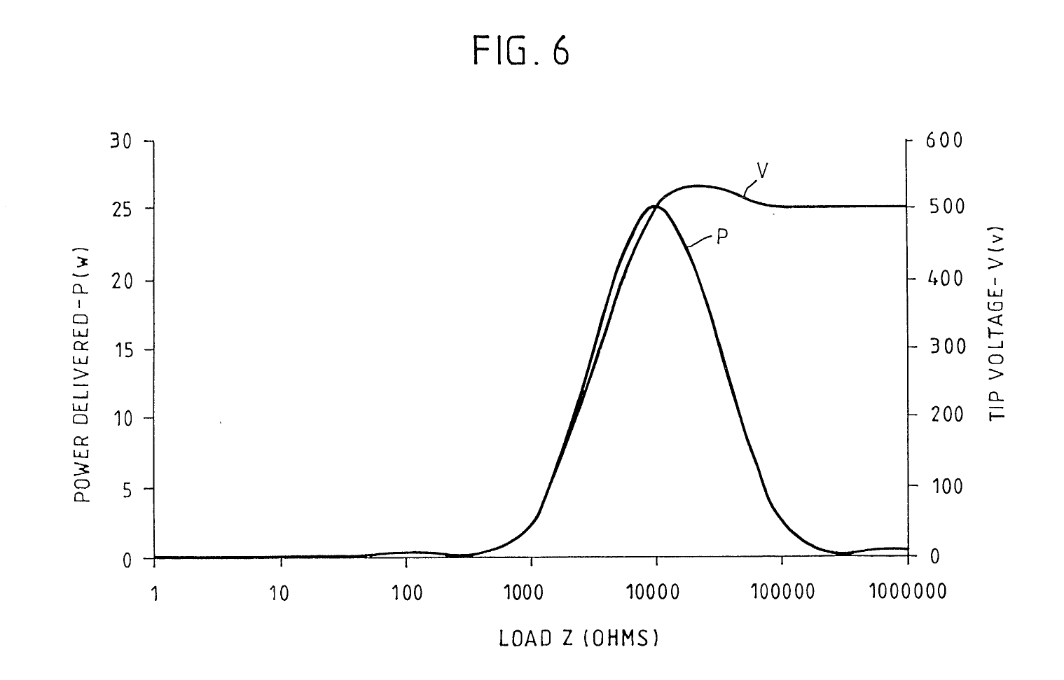

As will be seen from the graph of FIG. 6, the arrangement described above with reference to FIG. 5 yields maximum power transfer to the tissue when the tissue impedance is in the region of 10 k ohms. At 1 k ohm and below, both the delivered power and the output voltage are comparatively low, representing a stall condition. Stalling occurs, typically, when the electrode assembly encounters an electrolyte, such as when a blood vessel is cut. This condition is detected in a manner which will now be described.

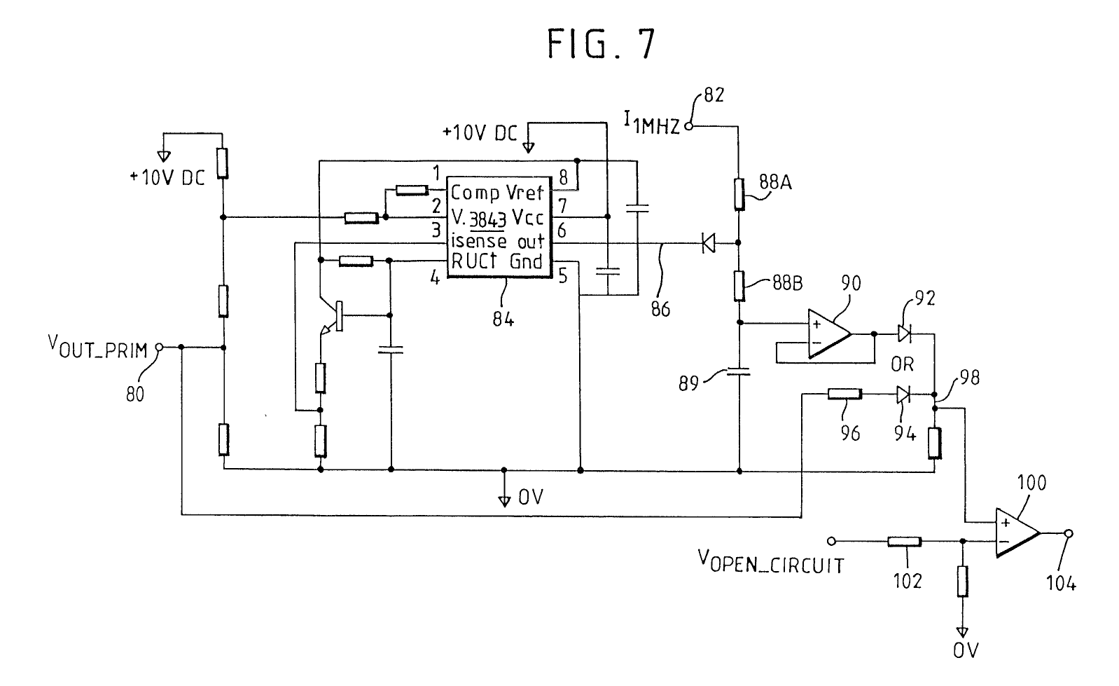

Referring to FIG. 7, a 1 MHz stall detector, forming part of the 1 MHz synthesiser 50 shown in FIG. 4, has voltage and current inputs 80 and 82 respectively. In the first instance, the stall detector applies the voltage from the primary winding of the transformer 66 (see FIG. 5) to a pulse width modulation chip 84 to produce a pulsed output signal having a pulse width which varies according to the voltage supplied at input 80. At input 82, a voltage proportional to the current in the primary winding of transformer 66, as sensed by the current transformer 70, is supplied to a potential divider 88A, 88B, the tap of the divider being connected to the output line 86 of the pulse width modulation chip 84. Accordingly, the voltage applied to buffer circuit 90, smoothed by capacitor 89, is equivalent to the pulse width modulation output on output line 86, scaled according to the level of the transformer primary current. In other words, the signal applied to buffer 90 represents the product of the transformer primary voltage and primary current, i.e. the delivered power at 1 MHz.

Thus, the signal at the output of buffer 90 is proportional to power, and is delivered to one input of an OR-gate formed by diodes 92, 94 which receives, at its other input, the voltage applied to input 80. Accordingly, the signal at the output 98 of the OR-gate is low only when both the delivered power at 1 MHz and the output voltage at 1 MHz are low, i.e. in accordance with the power and voltage characteristics shown in FIG. 6 when the load impedance is less than a few kilohms, and typically less than 1 k ohm. An output comparator circuit 100 is used to compare the output voltage from the OR-gate 92, 94 with a reference voltage applied to input 102, representing a reference value of the voltage obtained from the push-pull pair 64 (See FIG. 5) in open-circuit conditions. The resulting output at the detector output 104 is a control signal for enabling the 2.45 GHz synthesiser 52 shown in FIG. 4.

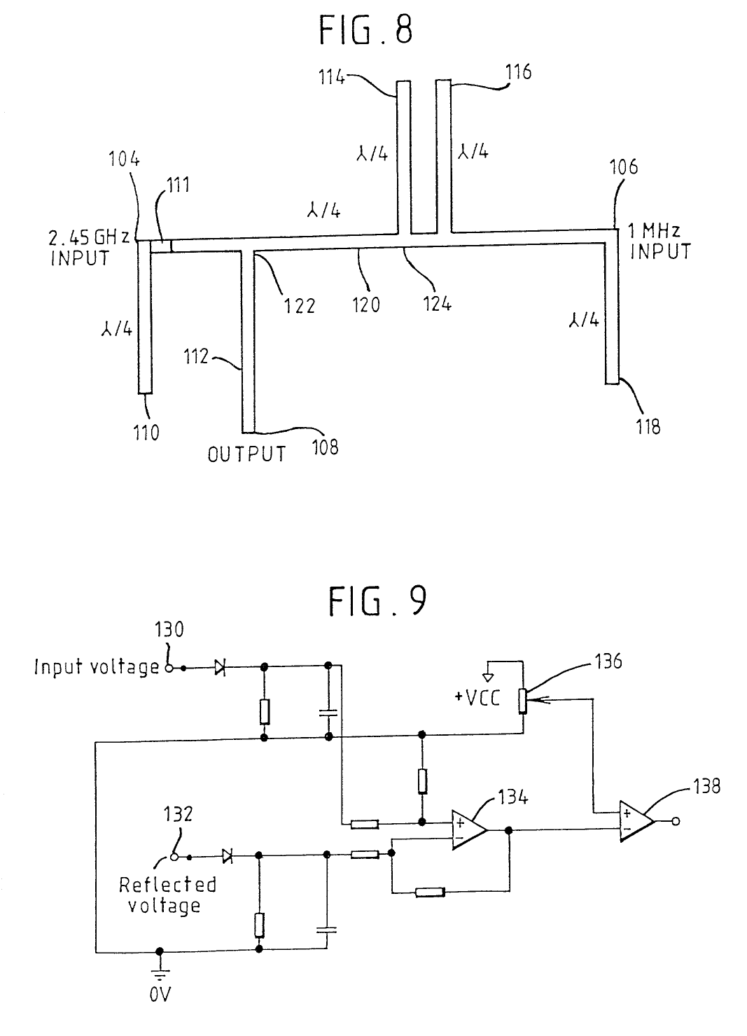

The adder 54 is formed as a microstrip device, as shown in FIG. 8. This is a 3-port device having a first input port 104 for the UHF signal from the 2.45 GHz generator part and a second input port 106 for the low frequency signal from the 1 MHz generator part. The device allows the UHF signal to be transmitted to an output port 108 with little loss whilst being isolated from the low frequency input port 106. Similarly, the low frequency signal applied to port 106 is transmitted to the output port 108 with low loss whilst being isolated from the UHF input port 104 a quarter wave (λ/4) short circuit stub 110 and series capacitor 111 at the UHF input port 104 are transparent to the signal applied at input port 104, which is thereby transmitted to the output port 108 via an output limb 112. Between the output limb 112 and the low frequency input 106 are three λ/4 open circuit stubs 114, 116, 118, the first 114 of these being spaced from the output limb 112 by a series λ/4 section 120. These open circuit stubs 114, 116, 118 reactively attenuate the 2.45 GHz signal to isolate it from the low frequency input 106. The base of the output limb 122 constitutes a sum injunction 112 and the λ/4 length of the line section 120 extends from this junction 112 to the base 124 of the first open circuit stub 114.

The open circuit stubs 114, 116, 118 are transparent to the 1 MHz signal, whereas the series capacitor 111 and the short circuit stub 110 reactively attenuate the 1 MHz signal in order to isolate the UHF input port 104 at 1 MHz.

It will be appreciated that the λ/4 components described above may, instead, have an electrical length which is any odd-number multiple of λ/4. Here, 2 is the wavelength of the applied UHF (2.45 GHz) signal in the microstrip medium.

The 2.45 GHz synthesiser includes a power control circuit as shown in FIG. 9. Referring to FIG. 9, the power control circuit has two inputs 130, 132 coupled to the input and the “reflected” power output of the circulator 56 (see FIG. 4) respectively. The reflected voltage applied to input 132 is subtracted from the input voltage supplied to 130 in comparator 134 and the resulting difference value compared with a reference voltage set by potentiometer 136 in an output comparator 138 to produce a switching signal for limiting the power output to a threshold value set by the user (or set automatically using a microprocessor controller forming part of the supply unit). Different power settings may be used depending upon the size of the electrode assembly connected to the handpiece and environment.

It will be appreciated that electrosurgical power may be delivered from the supply unit 10 shown in FIG. 1 either exclusively at 1 MHz or exclusively at 2.45 GHz for predominantly tissue vaporisation or thermal tissue coagulation respectively. In addition, power may be delivered at both frequencies simultaneously on the basis of a user-defined combination depending upon the characteristics of the tissue being treated. A third mode of operation is an auto-detection mode using the stall detection circuit described above with reference to FIG. 6, such that either of the two components predominate in a composite output voltage waveform, according to tissue impedance. In the latter case, the user typically selects a tissue vaporisation mode for predominant tissue cleaving or vaporisation, in which mode the 2.45 GHz component is enabled only when the tissue being treated presents a very low impedance. As mentioned above, this typically indicates the presence of an electrolyte such as blood from a blood vessel. Under these circumstances, the UHF component (i.e. the 2.45 GHz component) of the composite voltage waveform provides coagulation and/or desiccation of the tissue in the region of blood loss, the generator continuing in that mode until the detected tissue impedance rises again, whereupon the UHF component is disabled and treatment continues again exclusively at 1 MHz.

As described above, detection of low tissue impedance in these circumstances can be achieved by comparison of voltage and current amplitudes at the output of the 1 MHz source, prior to the adder 54 shown in FIG. 4. To avoid a low impedance detection output occurring as a result of reactive loading between the generator and the tissue being treated, the detector circuit may be modified to generate a signal representative of (V cos φ)/I, where V is the magnitude of the 1 MHz voltage component, I is the magnitude of the 1 MHz current component, and φ the phase angle between the said voltage and current.

It should be noted that detection of low power delivery at 1 MHz as described above with reference to FIG. 7 makes use of a signal representative of the real power delivered to the load, scaled by the voltage that would be obtained from the 1 MHz synthesiser with an open circuit output.

In an alternative embodiment, not shown in the drawings, the UHF (2.45 GHz) synthesiser 52 shown in FIG. 4 may be installed in the handpiece 12 together with the circulator 56, energy dump 58, and adder 54. This has the advantage that the cable 14 (see FIG. 1) between the supply unit and the handpiece 12 may be an inexpensive smaller diameter component. A d.c. power supply for the UHF synthesiser is also required, and may be provided by an additional cable or additional wires in the 1 MHz feed together with, when necessary, a further line for control functions. The composite output voltage is, in this case, fed directly from the adder 54 to the feeder structure represented by the instrument shaft.

It will be appreciated that losses at UHF are much reduced with this embodiment, to the extent that the power output of the UHF synthesiser may be reduced. Drawbacks include the additional bulk and weight of the handpiece and the possible need for forced fluid cooling of the UHF synthesiser, depending on the required power output. Such cooling could take place by evacuating air from the operation site into a passage at the distal end of the electrode shaft through a filter element to the UHF synthesiser, performing the dual functions of cooling the synthesiser and removing smoke or vapour from the operation site to enhance visibility.

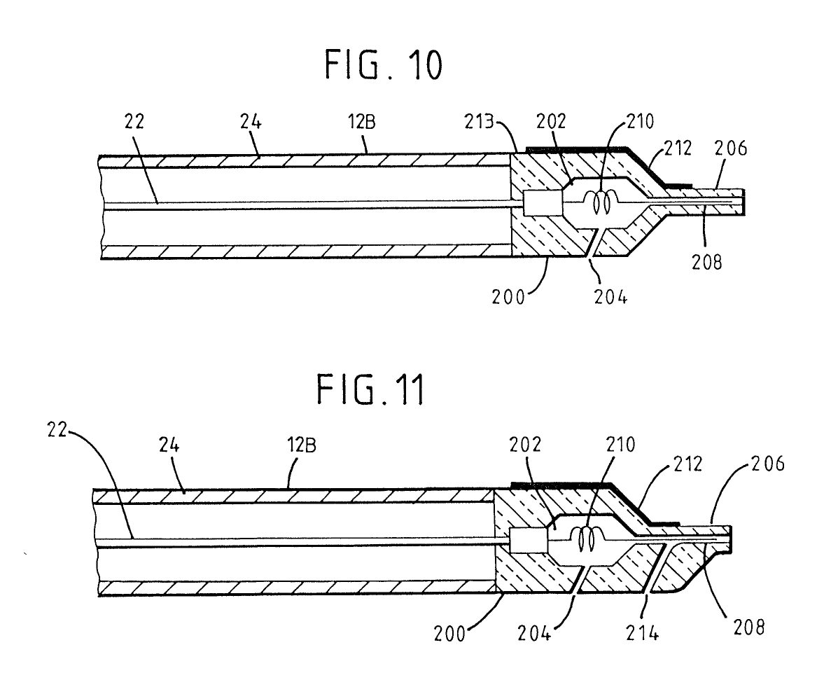

The ability to supply electrosurgical voltages at widely spaced frequencies also has application in a further alternative embodiment making use of a gas plasma electrode, as will now be described with reference to FIG. 10.

It is well known to use an inert gas such as argon, ionised using an r.f voltage and fed via a nozzle, typically having a diameter in excess of 1 mm, to produce a hot plasma “beam”. Directing this gas plasma onto the tissue being treated causes coagulation through transfer of thermal energy.

The behaviour of the argon plasma depends upon the incident energy. The higher the temperature of the argon, the greater its electrical conductivity. Paradoxically, the more energy initially imparted to the plasma, the less is the energy absorbed by the plasma due to its lower electrical impedance.

Supplying upper and lower frequency components simultaneously to a plasma-generating electrode assembly has the advantage that formation of the plasma can be performed independently of the conduction of energy along the plasma beam. As described above with reference to FIGS. 1 to 9, the upper and lower components typically have frequencies of 2.45 GHz and 1 MHz respectively.

Referring to FIG. 10, the preferred plasma generating electrode assembly consists of a ceramic nozzle body 200 attached to the end of a coaxial feed structure which has the same configuration as the feed structure in the embodiment described above with reference to FIGS. 1 to 9. Nozzle body 200 has an axial gas supply chamber 202 with a communicating lateral gas inlet 204. The nozzle body 200 is tapered distally to form a narrow tube 206 with an axial bore 208 providing an outlet from the chamber 202, the exit nozzle having an internal diameter in the region of 50 to 300 μm. Situated axially within the gas supply chamber 202 and the nozzle bore 208 is a whisker electrode 210 coupled to the inner supply conductor 22 of the coaxial feed. As shown in FIG. 10, the whisker electrode 210 is coiled within the chamber 202 and has an extension extending axially into bore 208 so that the total electrical length of the electrode 210 is about λ/4 at the frequency of the upper component.

Plated on the lateral exterior surface of the ceramic nozzle body 200 is a conductive return electrode 212 adjacent to the outer supply conductor 24 of the feed structure 12B and spaced from the supply conductor 24 by a gap 213.

Essentially then, the plasma generator comprises a whisker antenna within a ceramic tube having a metallised shroud. The capacitance between the whisker electrode 210 and the return electrode 212 is typically in the region of 0.5 to 5 pF. Clearly, this is a relatively low impedance at 2.45 GHz but a very high impedance at 1 MHz. This, coupled with the fact that the λ/4 length of the electrode 210 causes the electrode 210 to act as an impedance transformer producing a high voltage at the tip of the electrode, means that the 2.45 GHz component is dissipated within the plasma chamber when an ionisable gas is introduced via inlet 204 (causing plasma generation in bore 208) whereas the low frequency component at 1 MHz is conducted along the plasma beam to target tissue and to earth via the return pad attached to the patient (see FIG. 1).

The plasma generator is highly efficient at UHF frequencies, which means that the plasma may be generated with sufficient flow to absorb as much as 100 watts. The ionised gas is pumped from the chamber 202 through bore 208 which may have a bore as small as 0.1 mm. Since the majority of the power is dissipated within the chamber, little or no power at UHF is conducted to the nozzle outlet by the plasma. Instead, the UHF current component flows from the whisker electrode 210 via capacitive coupling to the return electrode 212, and thence via further capacitive coupling to the outer conductor 24 of the feed structure 12B.

Using the UHF source alone, the plasma beam acts as a powerful tissue coagulation tool, the depth and area of the coagulation effect being determined by the dispersion of the gas beyond the nozzle which depends, in turn, upon the distance the nozzle is held from the tissue surface. This is a purely thermal effect.

As described above, when both lower and upper frequency components are supplied, the lower frequency component at medium frequencies such as 1 MHz (a range of 100 kHz to 5 MHz is applicable in this instance) results in power being conducted along the plasma beam to the target tissue and thence to earth, vaporising the tissue.

Since the 1 MHz component is not coupled in plasma generation, its voltage can be comparatively low, at typically 300 volts to 1000 volts r.m.s. It follows that the ability of the low frequency source to support significant current delivery at low power is superior to that achievable in known prior systems.

The ionising ability of the UHF source is such that gases other than argon may be used. Argon has tended to be used in the prior art because it has a low ionisation potential, it is an inert gas, and it is the most abundant of the noble inert gases and consequently the cheapest. However, when using the described electrode assembly, with the plasma beam acting as an active electrode conveying electrosurgical tissue vaporising power at 1 MHz, a significant amount of residual carbon can be produced. This is the result of vaporising the tissue in an oxygen-free environment Use of an oxidising gas plasma by supplying oxygen or an oxide of nitrogen, gases which are both readily available in an operating theatre, counters the formation of carbon. Such gases have a considerably higher ionisation potential than argon with the result that considerably higher temperatures are attained with sufficiently conductive plasma streams, to the extent that the gas delivery rate has to be correspondingly reduced. An oxidising gas can be mixed with the argon before plasma generation, and introduced directly via inlet 204. Alternatively, the oxidising gas may be mixed with the argon plasma using an electrode assembly having a second gas inlet, as shown in FIG. 11. The embodiment shown in FIG. 11 makes use of a ceramic body 200 with a second lateral gas inlet 214 communicating with the bore 208 of the nozzle tube 206.

The whisker electrode 210 is preferably tungsten or tantalum due to the high melting point of these metals. Where an oxidising gas is introduced into the plasma generating chamber, a platinum or platinum-coated electrode is more appropriate, in order to avoid electrode oxidisation. The electrode may also be constructed from a thoriated alloy such as a thorium-tungsten alloy to improve electron emission and to promote predictable ionisation.

Dual frequency operation of a gas plasma electrode assembly as described above avoids the difficulties created by generating the plasma and the tissue effects from the same electrical source. Consequently, the difficulty in generating a plasma from a voltage which varies due to large variations in load impedance is avoided, and the lower frequency r.f. source can be used to deliver current through the plasma without relatively high peak voltages when using low frequencies, which places high power demands upon the r.f. generator. Narrow jet diameters, as disclosed above, as allowed by high excitation voltages and low impedance, result in higher current density upon tissue contact, giving the opportunity to perform rapid but fine tissue vaporisation.

The system described above with reference to FIGS. 1 to 9 may be modified to yield a high source impedance at the active electrode for the low frequency component, which yields further advantages.

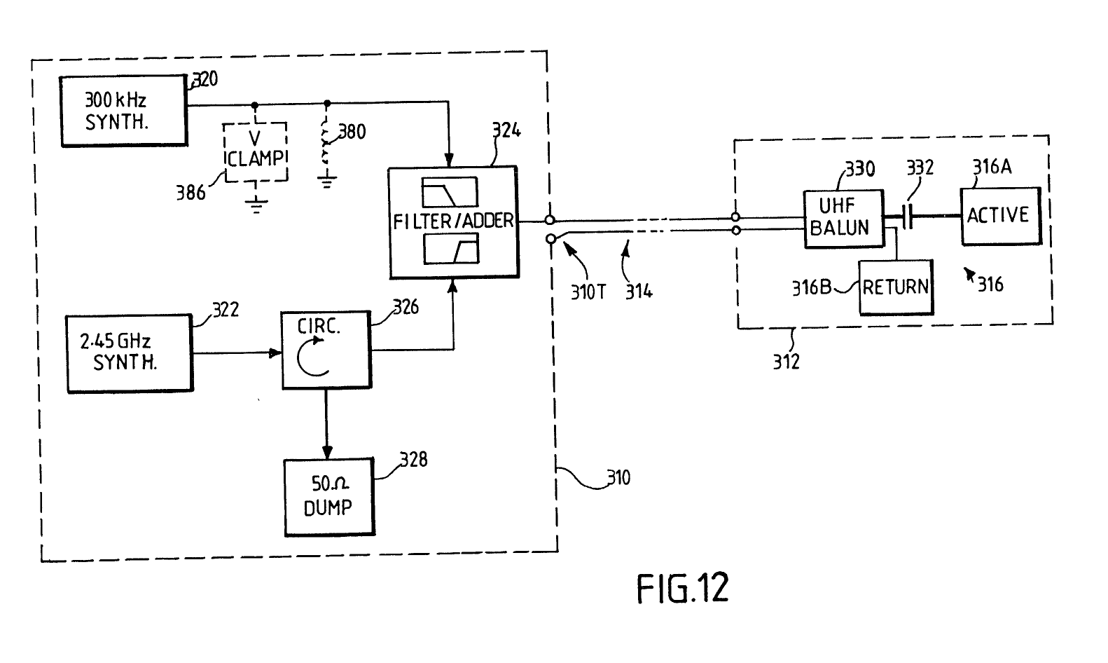

Referring to FIG. 12, a modified electrosurgical system in accordance with the invention comprises a dual-frequency generator unit 310 having output terminals 310C providing a radio frequency (r.f.) output to an electrosurgical instrument 312 via a flexible coaxial cable 314. The instrument 312 is in the form of a handpiece (not shown) with an instrument shaft having an electrode assembly 316 at its distal end, the assembly comprising the combination of an active or treatment electrode 316A and a return electrode 316B. The construction of the electrode assembly will be described hereinafter. It will be appreciated that in some embodiments, all or part of the generator unit may be incorporated within the handpiece. Whether it is in the handpiece or separate, the generator may be activated by a switch in the handpiece or a foot switch separately connected to the generator unit 310. The mode of operation, e.g. coagulation, cutting and vaporisation modes, is selected by controls also not shown in FIG. 12.

The generator unit 310 contains separate 300 kHz and 2.45 GHz synthesisers 320, 322 the output signals of which are summed in adder 324 having low- and high-pass filters coupled to inputs arranged to receive the 300 kHz and 2.45 GHz signals respectively as shown. A circulator 326 connected in series between the 2.45 GHz synthesiser 322 and the adder 324 serves to provide a 50 ohm source impedance for synthesiser 322 under conditions of varying load impedance, with effective power being dissipated in a 50 ohm reflective energy sink or dump 328, also connected to the circulator 326.

At the output of the adder 324, a composite signal consisting principally of the two frequency components at 300 kHz and 2.45 GHz is delivered to the output terminals 310T of the generator unit 310 and fed via a cable 314, which is typically in the region of 3 m long, to the handheld instrument 312 and thereafter to the tissue under treatment. Both low and high frequency components are, consequently, fed via a single feeder structure to the electrodes 316A, 316B The instrument 312 also includes a UHF balun 330 for converting the high frequency (i.e. 2.45 GHz or UHF) component from a single-ended signal, as present at the output terminals 310T of the generator unit 310, to a balanced signal at the active and return electrodes 316A and 316B. During operation of the system, r.f. energy is delivered by the generator unit 310 along the inner conductor of the feeder cable 314 via balun 330 to the active electrode 316A. The current then passes from active electrode 316A through the tissue being treated, and back via the capacitance between the tissue and the return electrode 316B to the generator along the outer conductor of the feeder cable 314. Included in this current path is a current limiting capacitor 332 which raises the source impedance in respect of the low frequency (300 kHz) component as seen at the electrodes. In the present embodiment, this capacitor has a value in the region of 1.5 pF and is located immediately adjacent the active electrode 316A at the distal end of the instrument shaft. In other embodiments it may be located elsewhere in the current path between the low frequency source 320 and the electrodes 316A, 316B, but the position at the distal end of the shaft is preferred to avoid the shunt capacitive loading of the instrument shaft and/or the feeder cable 314.

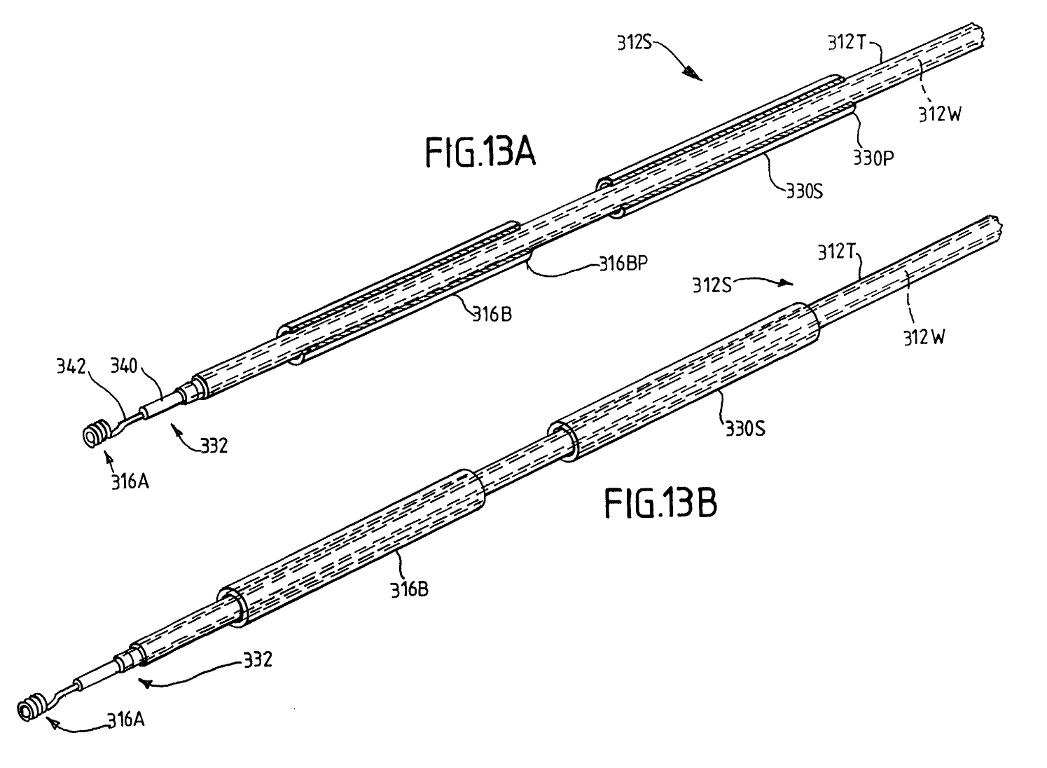

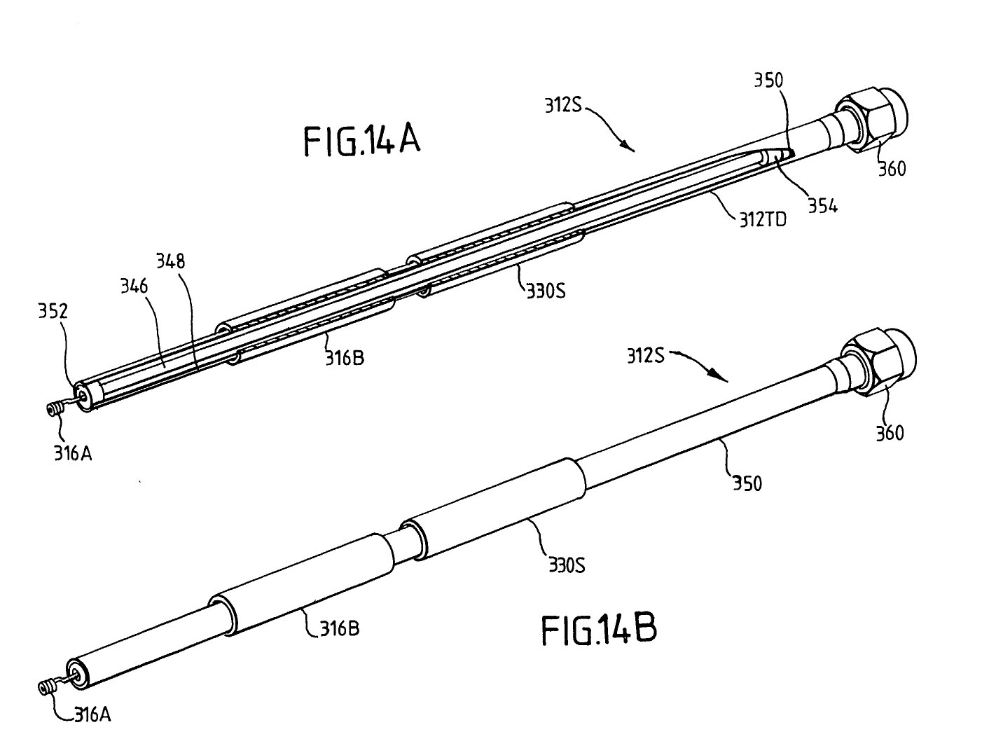

A distal end portion of the instrument shaft is shown in FIGS. 13A and 13B. Referring to these figures, shaft 3112S takes the form of a rigid stainless steel tube mounted at its proximal end in a handpiece body (not shown). The shaft 312S constitutes a coaxial feed structure, with the stainless tube 312T acting as an outer supply conductor 312T. An inner wire 312W, insulated from the tube 312T via an insulating sleeve (not shown) forms an inner conductor. This inner conductor is tubular at the distal end of the shaft, where it is in the form of metallisation on a narrow ceramic tube 340, part of which is exposed beyond the distal end of outer conductor 312T, as shown as FIGS. 13A and 13B. Fixed within tube 340 is a central wire 342 the end of which, in this embodiment, is coiled to form an active electrode 316A suitable for tissue vaporisation. The ceramic material of tube 340 constitutes a low loss ceramic dielectric of a tubular capacitor formed by the metallisation on the tube 340 and the central wire 342. This capacitor has a value of about 1.5 pF and, as such, represents a significant series impedance at the low operating frequency of 300 kHz but at the upper frequency of 2.45 GHz its impedance is comparable to or lower than the typical load impedance represented by the tissue under treatment and the capacitative return path.

The balun 330 is created by a conductive sleeve 330S around the coaxial feed structure, the sleeve having an electrical length of λ/4 and connected at its proximal end 330T to the outer supply conductor formed by tube 312T.

The return electrode 316B is in the form of a similar conductive sleeve, also connected at its proximal end 316BP to the outer supply conductor. Both the balun sleeve 330S and the return conductor are quarter-wave resonant structures located on the distal end portion of the shaft 312S. The complete shaft and these sleeves are covered by an insulating layer which is not shown in FIGS. 13A and 13B.

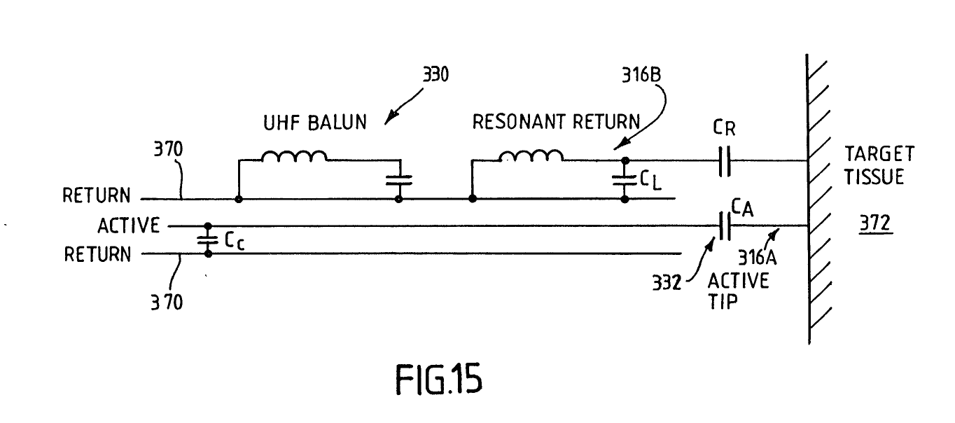

An alternative configuration for the distal end of the shaft 312S is shown in FIGS. 14A and 14B. In this case, the current limiting capacitor (shown as element 332 in FIG. 12) has an air dielectric, being formed by the combination of an axial conductive rod 346 and the inner metallisation 348 of a rigid insulative tube 350 which is also metallised on the outside to form the outer supply conductor 312TD of the shaft distal end portion. Inner rod 346 is held in its axial position by insulative spacers 352, 354. At its distal end, the inner rod 346 is connected to a wire electrode 316A which, in this case, is somewhat smaller than the active electrode of the embodiment of FIGS. 13A and 13B, and is more suitable for tissue cutting. The rod 346 terminates at the proximal spacer 354 and the inner metallisation of tube 350 is connected to the inner supply conductor of a coaxial connector 360, while the outer metallisation on tube 350 is connected to the connector outer shield so that the shaft portion shown in FIGS. 14A and 14B may be connected to a proximal coaxial shaft portion, or directly to a handpiece body (neither shown). The balun sleeve 330S and the return electrode 316B are similarly constructed and connected as the equivalent components of the embodiments of FIGS. 13A and 13B and, again, the complete assembly is covered with an insulative coating, with the exception of electrode 316A.

As an aid to understanding the operation of the system, attention is directed to the equivalent circuit of FIG. 15, the cutaway sleeve 330S that creates the quarter wave sleeve balun being represented by a lumped inductor and capacitor combination connected to the outer supply conductor of the shaft 312S, here designated the “return” conductor 370. This balun matches inner and outer UHF currents. The return electrode sleeve 316B is also shown as a lumped resonant structure. This operates in a similar fashion to the balun but provides the predominant return path for r.f. energy at UHF, the resonant structure amplifying the return voltage due to its resonance at the upper operating frequency of 2.45 GHz. The inductance of the return electrode sleeve 316B has a value such that it resonates with the combination of the stray return capacitance CR and sleeve-to-shaft capacitance CL at 2.45 GHz. The return electrode is dimensioned accordingly.

It will be appreciated that the circuit elements due to the balun and return electrode sleeves 330, 316B are effectively invisible at the lower operating frequency. However, the current limiting capacitance 332 and the feeder capacitance Cc, which appears as a lumped capacitance at the lower frequency, have a significant effect. The value of capacitor 332 is typically 1.5 pF, this value being appropriate for a lower operating frequency of about 300 kHz. Alternative values having an equivalent series impedance may be selected for different lower operating frequencies. The effect of capacitor 332 is to limit the lower frequency current delivery to inconsequential values in terms of clinical effect.

When the system is used for tissue vaporisation, the active tissue 316A can become hot. In such circumstances, it is possible for thermionic rectification to occur, causing a charge build-up on any coupling capacitance such that intermittent contact with tissue subsequently causes alternate charging and discharging of the coupling capacitor. Positioning the capacitor 332 directly adjacent active electrode 316A allows it to remain small in value so that nerve stimulation due to thernionic rectification is virtually absent.

The capacitance Cc of the cable represents a low impedance source at the lower operating frequency and in this context coupling capacitor 332 has the advantage of reducing any high current discharge through an arc established between the active electrode tip 316A and the target tissue 372 due to the feeder capacitance Cc.

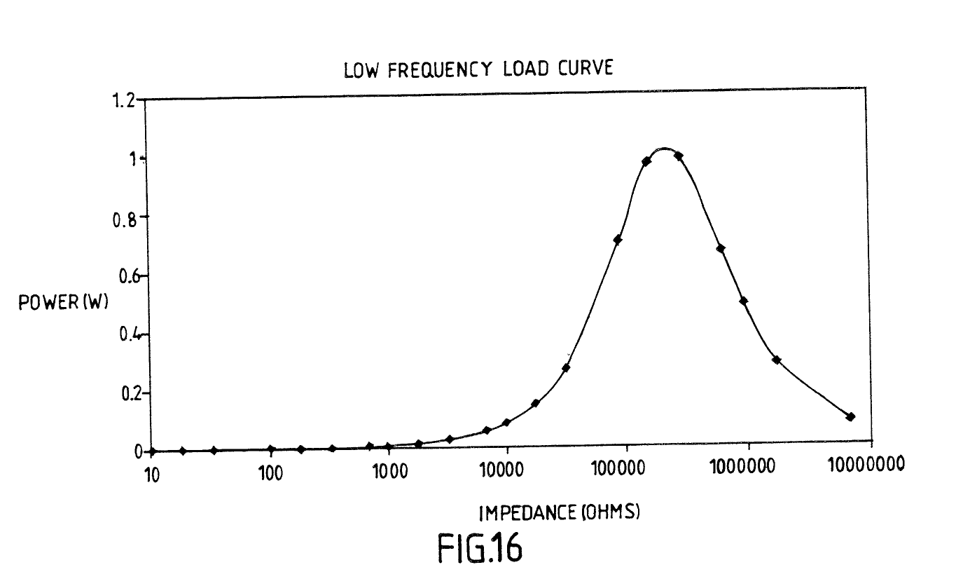

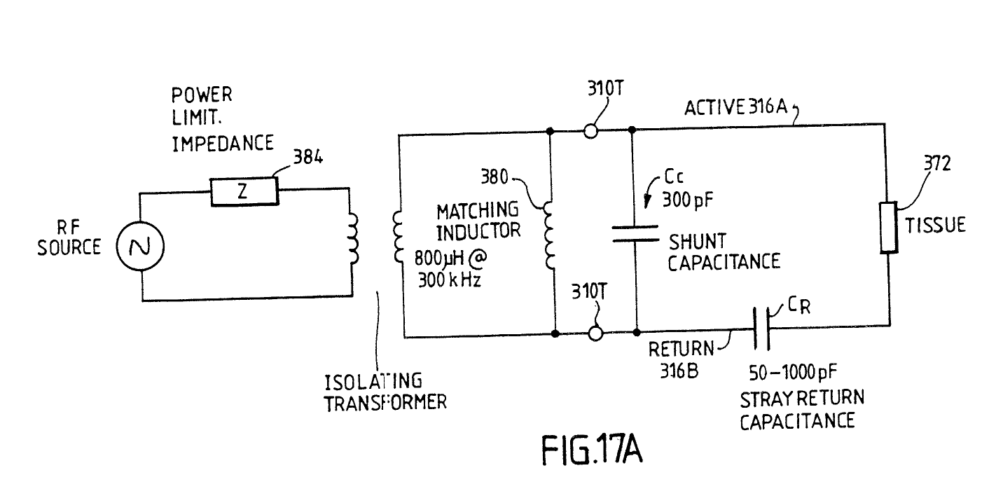

The raising of the source impedance at the lower operating frequency due to the coupling capacitor 332 is illustrated in the power/impedance load curve of FIG. 16 which indicates maximum power occurring at about 250 kilohms, the effective source impedance.

As mentioned above, the effect of the coupling capacitance 332 allows a high voltage low frequency signal to be applied across the electrodes 316A, 316B without giving rise to corresponding currents at the lower frequency which have the potential to cause tissue effects both at the treatment site and at other sites on the patent's body, e.g. along luminal structures such as blood vessels or adjacent an earthed structure such as an operating table. Accordingly, in a tissue cutting or vaporisation mode of the system, the 300 kHz synthesiser 320 (FIG. 12) can be activated to provide sufficient voltage across the electrodes 316A, 316B to cause arcing when the active electrode 316A is close to the target tissue 372. Simultaneous application of the 2.45 GHz and 300 kHz components to the tissue 372 allows UHF current to flow from the active electrode 316A along the arc to the tissue. Return currents of both components are coupled to the return electrode 316B by the stray tissue-to-electrode capacitance CR. The current path provided by the arc constitutes a comparatively low impedance at UHF which means that the load impedance in the cutting or vaporisation mode is comparable to that in the coagulation mode. Accordingly, the same system may be used for both coagulation and cutting/vaporisation, taking advantage of the localisation of effect which can be achieved at UHF when driving impedances below 1 kilohm.

The level of voltage applied at the lower operating frequency to initiate an arc may be as low as 300 V peak. A voltage in excess of 1000 V peak may be used for tissue vaporisation. Once initiated, it is possible to sustain an arc with a voltage of less than 100 V peak. The low impedance pathway created by the arc exists only for a very short time, but this is sufficient for coupling of UHF energy along the same pathway, high UHF currents being possible due to the considerably lower impedance of the return pathway at UHF. Should the active electrode 316A contact the target tissue 372, the applied voltage at the lower operating frequency will collapse so that a very small maximum current is delivered. Formation of the arc causes instantaneous discharge of the coupling capacitor 332 resulting in a very brief high current impulse which has a peak power much higher than the peak power available from the UHF source and which is capable of exciting the resonance of the resonant circuits represented by the return electrode 316B and balun 330 located at the distal end of the instrument shaft. These factors ensure that low frequency arcing provides a conductive pathway for the UHF component.

Vaporisation can be initiated in two ways. If the active electrode 316A is brought into close proximity with the tissue 372 such that the low frequency component initiates an arc, the ionised pathway is then the preferred path for UHF current. Since the ionised pathway is extremely narrow at any instant, the subsequent delivery of UHF is with very high power density, which is capable of vaporising tissue. The ionised pathway moves towards the closest conduction point, with the result that all tissue within the arc strike distance of the active electrode 316A is vaporised. The second method of arc initiation is with the active electrode 316A already in contact with the tissue. Initially, the low frequency component is stalled by low impedance contact, but the delivery of UHF power through the low impedance contact results in tissue coagulation and desiccation. Desiccation proceeds until the electrode-to-tissue impedance rises sufficiently to allow a low frequency voltage gradient between the electrode and the tissue for creating the arc (the impedance at that point being greater than 50 kilohm).

The advantages of this method of operation are that all r.f. power is localised to the treatment zone, and the structure of the electrode assembly need be configured only for low impedance (high current) UHF power delivery. Such UHF power delivery may be optimised for tissue contact coagulation, cutting and vaporisation being achieved by addition of the low frequency component. Further advantages are the ability to use only low power low frequency drivers, much reduced radio frequency emissions due to 110 the avoidance of currents through an earth return pad, and the ability to adjust the effect (e.g. between cutting and different degrees of vaporisation) by adjusting the low frequency peak voltage and the consequent arc striking distance. The ability to provide low frequency coupling by a comparatively small capacitance yields the advantage that stray return capacitance effects are negligible.

While some of the advantageous effects of situating the coupling capacitor in an electrode assembly may be lost, it is possible to achieve arc initiation with alternative capacitor positioning. For instance, the capacitor can be located in the handpiece body, i.e. at the proximal end of the instrument shaft, in which place a capacitance value in the range of from 20 pF to 100 pF is appropriate. It is also possible to locate the capacitor in the generator unit. In this case, where a feeder cable is present, an appropriate capacitor value would be of the range of 300 pF to 1 nF.

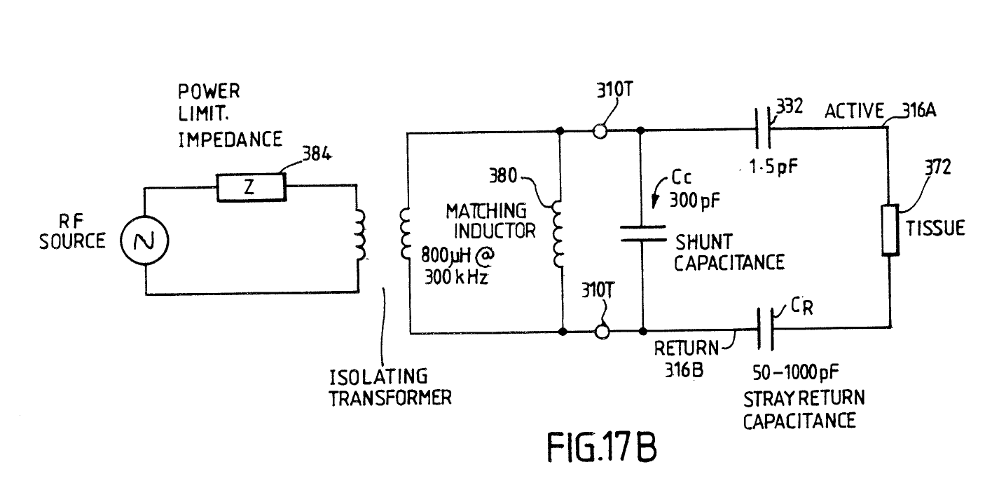

Current limiting at the lower operating frequency may be achieved by alternative means. As an example, current limiting may be performed by the combination of low power delivery at the lower operating frequency in conjunction with resonant impedance transformation. The coupling capacitor 332 of the above-described embodiment may be omitted. Referring, then, to FIG. 17A, the capacitance Cc of the feeder between the generator unit 310 and the active and return electrodes 316A, 316B is typically in the region of 300 pF. This typically sets the low frequency source impedance as seen at the electrodes 316A, 316B to a value below 10 kilohms. At 300 kHz, 300 pF represents an impedance of 1.77 kilohms. To achieve similar steady state limiting as with the coupling capacitor embodiment described above, the impedance may be converted to a value above 100 kilohms, typically 250 kilohms, by use of a matching inductor 380 (see FIG. 12 as well as FIG. 17A) which forms a resonant circuit with the feeder capacitance Cc at the lower operating frequency, it being understood that in this case, capacitor 332 is omitted. At 300 kHz, the value of the matching inductance required to match out the 300 pF capacitance Cc of the feeder is about 800 μH. The Q of the resonant circuit is preferably greater than 100 and typically greater than 140. This yields a source impedance of about 250 kilohms and has a similar effect on current delivery as that produced by the coupling capacitor 332 of the previous embodiment. Power delivery at the lower operating frequency is limited to 20 W or less, typically less than 5 W, by a series impedance 384 in the low frequency part of the generator unit upstream of the combiner 324 (see FIG. 12). Again, only a low power low frequency driver is necessary. Potentially, the peak energy associated with arc initiation is higher in this embodiment due to shunt capacitance Cc of the feeder being directly coupled to the electrodes 316A, 316B, with the result that the arc pathway has a lower impedance. To maintain the operating frequency of the lower frequency component at or near the resonant frequency of the combination of the cable capacitance Cc and the inductance 380, the 300 kHz synthesiser 320 is configured to track the resonance of the resonant circuit by self-tuning oscillation, as disclosed in U.S. Pat. No. 5,099,840, or by means of a closed loop control system using current and voltage phase relationships to alter frequency, as disclosed in U.S. Pat. No. 6,093,186. The contents of these patents are incorporated in the disclosure of the present application by reference. Other methods of achieving frequency tracking are known in the art.

The rapidity with which arc strikes can be initiated using the resonant circuit technique of lower frequency current limiting may be increased by modulating the 300 kHz synthesiser output. For instance, if the output of this synthesiser is pulse modulated with a 50% duty cycle, the driving impedance of the r.f. source into the resonant network (inductor 380 and the feeder capacitance Cc) may be halved, since the average low frequency current compared with continuous delivery at the higher drive impedance is maintained. Consequently, the low frequency output voltage is correspondingly higher than required to initiate arcing, with the effect that an arcing voltage is reached more quickly. Limiting of the voltage may be performed by a voltage clamp shown in FIG. 12 by element 386 using either zener diodes, varistors, or a variable active clamp such as well known in the art. The modulation duty cycle is preferably greater than 10% to reduce the likelihood of the maximum peak current reaching a value liable to cause the peak voltage developed between the patient and the ground to rise above 300 V.

The series impedance 384 and resonating inductor 380 may be used in conjunction with the coupling capacitor in the electrode assembly, as shown in FIG. 17B.

What is claimed is:

1. An electrosurgery system for electrosurgically cutting or vaporising living tissue, comprising an electrosurgical generator and an electrode assembly having at least one treatment electrode and an adjacent return electrode, wherein the generator and the assembly are arranged to deliver to the treatment and return electrodes radio frequency (r.f.) energy simultaneously at at least two frequencies, one of which is in a lower frequency range of from 50 kHz to 50 MHz and the other of which is greater than 300 MHz, the r.f. current delivered in the lower frequency range being limited such that the current-to-frequency ratio of energy delivered in the lower frequency range remains below a value of 17 mA r.m.s. per 100 kHz.

2. A system according to claim 1, arranged to deliver the said r.f. energy to the electrode assembly at both of the two frequencies along a single feeder between the generator and the electrodes.

3. A system according to claim 1, comprising a generator unit having a pair of r.f. output terminals, an electrosurgical instrument which includes a handpiece, a shaft mounted in the handpiece and the electrode assembly located at a distal end of the shaft, and a feeder cable arranged to connect the generator unit output terminals to the handpiece, wherein the instrument includes a current limiting capacitor connected in series between the feeder cable and the treatment electrode for limiting the current at the lower frequency to the said current range.

4. A system according to claim 3, wherein the capacitor is located at the distal end of the shaft.

5. A system according to claim 4, wherein the shaft comprises at least a pair of supply conductors for delivering the r.f. energy to the electrode assembly, and wherein the capacitor is formed as the coaxial combination of an elongate inner conductor, a tubular heat-resistant dielectric tube around the inner conductor, and a tubular outer conductor around the dielectric tube, one of the said inner and outer conductors of the combination being connected to one of the supply conductors of the shaft and the other being connected to the treatment electrode.

6. A system according to claim 5, wherein the treatment electrode is monolithically integral with the capacitor inner conductor.

7. A system according to claim 3, wherein the capacitor has a value of 5 pF or less.

8. A system according to claim 3, wherein the capacitor is located in the handpiece and has a value in the range of from 20 pF to 100 pF.

9. A system according to claim 1, comprising a generator unit having a pair of output terminals, an electrosurgical instrument which includes a handpiece, a shaft mounted in the handpiece, and the electrode assembly located at the distal end of the shaft, and a feeder cable arranged to connect the generator unit output terminals to the handpiece, wherein the system includes a low frequency source to generate r.f. energy in the said lower frequency range, and a current limiting impedance coupled in series between the low frequency source and the feeder cable.

10. A system according to claim 9, wherein the current limiting impedance is a capacitor the value of the which is in the range of from 300 pF to 1 nF.

11. A system according to claim 1, including a balun associated with the electrode assembly, the balun being configured to operate at the said frequency greater than 300 MHz.

12. A system according to claim 1, having a handheld electrosurgical instrument which includes an elongate shaft mounted in the handpiece, and the electrode assembly located at a distal end of the shaft wherein:
the shaft comprises at least a pair of supply conductors forming a coaxial feeder structure for delivering electrosurgical r.f. energy from the generator to the electrode assembly;
the treatment electrode is electrically coupled to an inner supply conductor of the shaft;
the return electrode is electrically coupled to an outer supply conductor of the shaft and is set back from the treatment electrode;
the shaft carries a balun adjacent the electrode assembly, the balun being electrically coupled to the outer supply conductor; and
the shaft, the return electrode and the balun are covered in an insulative material.

13. A system according to claim 12, wherein the electrode assembly includes a current limiting capacitor in series between the inner supply conductor and the treatment electrode for limiting the current supplied to the electrodes at the lower frequency such that the said ratio remains within the said range.

14. A system according to claim 1, wherein the source impedance at the treatment electrode at an operating frequency in the lower frequency range is greater than 100 kilohm.

15. A system according to claim 1, wherein the current at an operating frequency in the lower frequency range is limited by means for increasing the source impedance at that frequency.

16. A system according to claim 15, wherein the current limiting means comprises a capacitance in series with the treatment electrode.

17. A system according to claim 16, wherein the current limiting means comprises a resonant impedance converter associated with an output of the generator.

18. A system according to claim 17, wherein the impedance converter comprises a parallel resonant circuit.

19. A system according to claim 17, including a coaxial feeder between the generator output and the electrode assembly, and wherein the impedance converter comprises an inductance associated with the generator output which resonates with the capacitance of the feeder at the operating frequency in the lower frequency range.

20. A system according to claim 19, wherein the generator is arranged such that the r.f. energy in the lower frequency range is pulse modulated.

21. A system according to claim 20, wherein the pulse duty cycle is at least 10%.

22. A system according to claim 1, arranged such that the peak voltage in the lower frequency range when in a cutting/vaporisation mode is in excess of 500 V.

23. A system according to claim 1, wherein the energy in the said lower frequency range is delivered at a frequency of 100 kHz or higher.

24. A system according to claim 1, wherein the energy in the said lower frequency range is delivered at a frequency of 5 MHz or below.

25. A system according to any preceding claim, wherein the r.f. current delivered in the lower frequency range remains below 50 mA r.m.s.

26. A method of operating an electrosurgical tissue cutting or vaporising system using an electrosurgical instrument having an active electrode and an adjacent return electrode, wherein the method comprises supplying to the electrodes radio frequency energy simultaneously at at least two frequencies, one of which is in a lower frequency range of 50 kHz to 50 MHz and the other of which is greater than 300 MHz, the current in the lower frequency range whilst the instrument is set to operate in a tissue cutting or vaporising mode being such that the current-to-frequency ratio of energy delivered in the lower frequency range remains below a value of 17 mA r.m.s. per 100 kHz.

27. A method according to claim 26, including maintaining the current-to-frequency ratio below the said value by driving the active electrode from a source impedance which is between 100 kilohm and 500 kilohm at the operating frequency in the lower frequency range.

28. A method according to claim 26, wherein the tissue cutting or vaporising mode is characterised by a peak voltage in the lower frequency range between 500 V and 2000 V.

29. A method of electrosurgically treating tissue using an electrosurgical instrument having an active electrode and an adjacent return electrode, comprising successively (a) cutting or vaporising tissue, and (b) coagulating tissue, wherein both steps (a) and (b) are performed by delivering radio frequency energy to the electrodes at a frequency greater than 300 MHz, and wherein step (a) is characterised by simultaneously supplying r.f. energy at a frequency within a lower frequency range of from 50 kHz to 50 MHz, the r.m.s. current in the lower frequency range being limited to a value such that the current-to-frequency ratio of energy delivered in the lower frequency range remains below 17 mA r.m.s. per 100 kHz.

30. A method according to claim 29, wherein the r.f. energy delivered in the lower frequency range is pulsed, and the current-to-frequency ratio of energy delivered within each r.f. pulse burst in the lower frequency range remains below the said current-to-frequency ratio value.

31. A system according to claim 30, wherein the r.f. current in the lower frequency range during the pulse bursts remains below 50 mA r.m.s.

32. A method of electrosurgically cutting or vaporising tissue using an electrosurgery system which comprises an electrosurgical generator and an electrode assembly having at least a treatment electrode and an adjacent return electrode, wherein the method comprises bringing the treatment electrode to a position on or adjacent the tissue to be cut or vaporised, applying to the electrodes a first radio frequency (r.f.) signal component at at least one frequency in the range of from 50 kHz to 50 MHz to establish an arc between the treatment electrode and the tissue, and simultaneously applying to the electrodes a second r.f. signal component at at least one second frequency which is greater than 300 MHz to cause a current at the second frequency to flow along the arc established by the first r.f. signal component, the level of the average current above 300 MHz being at least on order of magnitude greater than the average current in the frequency range of from 50 kHz to 50 MHz during a cutting or vaponsation operation.

33. A method according to claim 32, wherein the average current in the frequency range of from 50 kHz to 50 MHz is small enough to have no clinical effect or negligible clinical effect on the patient in the absence of the second r.f. signal component.

34. An electrosurgery system comprising an electrosurgical generator, a feed structure and an electrode assembly, the electrode assembly having at least one active electrode and at least one adjacent return electrode, each of which is coupled to the generator via the feed structure, wherein the generator and feed structure are capable of delivering radio frequency (r.f.) power to the active and return electrodes in lower and upper frequency ranges simultaneously, and wherein the lower frequency range is below 100 MHz and the upper frequency range is above 300 MHz.

35. A system according to claim 34, wherein the return electrode is an element which is resonant at an operating frequency in the upper frequency range.

36. A system according to claim 35, wherein the operating frequency is above 1 GHz.

37. A system according to claim 34, wherein the electrode assembly has associated therewith a sleeve balun operable at an operating frequency in the upper frequency range.

38. An electrosurgery system comprising an elecrosurgical generator and a handheld electrosurgical instrument, wherein the generator is capable of delivering to the instrument radio frequency power in lower and upper frequency ranges, the upper range containing frequencies at least three times the frequencies of the lower frequency range, wherein the instrument includes (a) an instrument shaft which comprises a coaxial feeder having an inner conductor and an outer conductor and (b) an electrode assembly at an end of the shaft, the assembly comprising a first electrode electrically coupled to the inner conductor and a second electrode in the form of a conductive sleeve set back from the first electrode and surrounding a portion of said outer conductor, and wherein the sleeve has an end portion which includes an electrical connection to said outer conductor, the remainder of the sleeve being spaced from said outer conductor.

39. A system according to claim 38, wherein the first electrode is capacitively coupled to said inner conductor.

40. A system according to claim 39, wherein the first electrode is coupled to said inner conductor by a capacitor which comprises an elongate coaxial assembly inside said feeder outer conductor.

41. A system according to claim 40, wherein the coaxial assembly comprises a solid dielectric tube containing an axial wire, the tube having an outer conductive layer.

42. A system according to claim 40, wherein the coaxial asembly comprises an axial rod and an insulative tube with an inner conductive layer, the rod supported coaxially within the tube and spaced from the inner layer.
